SEQUENCE LISTING (2 sequences)

SEQ ID NO: 1 - DNA, 20 bp, synthetic construct
  source 1..20: mol_type = other DNA
tgagattcctgggttcaagg

SEQ ID NO: 2 - DNA, 20 bp, synthetic construct
  source 1..20: mol_type = other DNA
gtcagaacactgcacgttgg

(19) United States
(12) Patent Application Publication
(10) Pub. No.: US 2025/0312466 A1
(43) Pub. Date: Oct. 9, 2025

(54) NUCLEIC ACID CLOSING POLYMERIC MICELLE

(71) Applicant: THE UNIVERSITY OF TOKYO, Tokyo (JP)

(72) Inventors: Horacio CABRAL, Tokyo (JP); Pengwen CHEN, Tokyo (JP); Wenqian YANG, Tokyo (JP); Rigte Boonstra EGER, Tokyo (JP)

(73) Assignee: THE UNIVERSITY OF TOKYO, Tokyo (JP)

(21) Appl. No.: 18/873,092

(22) PCT Filed: Jun. 12, 2023
(86) PCT No.: PCT/JP2023/022516

§ 371 Date: Dec. 9, 2024

Related U.S. Application Data
(60) Provisional application No. 63/351,358, filed on Jun. 11, 2022.

(51) Int. Cl. A61K 47/64; A61K 31/7088; A61K 47/59; A61K 48/00; C07K 2/00; C12N 15/11

(52) U.S. Cl. CPC A61K 47/6455; A61K 31/7088; A61K 47/59; A61K 48/0041; C07K 2/00; C12N 15/11

(57) ABSTRACT

The present invention provides a pH-responsive carrier for nucleic acid delivery to cells or tissues, comprising a combination of a cationic polymer having a side chain containing a primary amine and a block copolymer represented by the following formula (1):

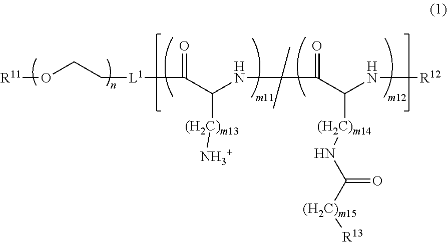

### TECHNICAL FIELD

The present invention relates to a nucleic acid-enclosing polymeric micelle which is configured to achieve improved stability in a severe in vivo environment by using a block copolymer. All disclosures of the references cited herein are incorporated herein by reference in their entirety.

### BACKGROUND ART

Since mRNA is translated into therapeutic proteins in the cytoplasm, it has a potential for nucleic acid therapeutics. While plasmid DNA, one of nucleic acid therapeutics, can induce insertion into the host genomic DNA and require delivery systems targeting to the cell nucleus, mRNA holds advantages over such plasmid DNA (U. Sahin et. al., Nat. Rev. Drug Discovery 13 (2014), 759-780). However, systemic administration of naked mRNA shows rapid enzymatic degradation due to negatively charged phosphate group, poor cellular uptake and unfavorable immune responses (N. B. Tsui et. al., Clin. Chem. 48 (2002), 1647-1653). Thus, the development of nanocarriers loading mRNA is essential for application of mRNA.

Polyion complex (PIC) micelles are one of the promising nanocarriers capable of delivering mRNA. Block copolymers comprising poly(ethylene glycol) and polycation can encapsulate mRNA via electrostatic interactions to protect mRNA payload in the PIC core ((S. Uchida et. al., Biomaterials 82 (2016), 221-228). mRNA-loaded PIC micelles comprising poly(amino acids) is utilized to inhibit enzymatic degradation of loaded mRNA, and to enhance the cellular uptake via charge neutralization, resulting in the achievement of augmented gene expression (S. Uchida et. al., Biomaterials 82 (2016), 221-228).

However, further improvement of PIC stability in harsh environment in vivo is important for in vivo applications.

### SUMMARY OF THE INVENTION

### Problem to be Solved by the Invention

Accordingly, for enhancement of the therapeutic effect provided by therapeutic nucleic acids, it is important to develop micelles which allow increased blood retention and efficient release of nucleic acids under acidic conditions.

### Means to Solve the Problem

The present invention aimed at increased stability of micelles and efficient release of a nucleic acids under acidic conditions by introducing a pH-responsive maleic anhydride derivative and cationic polymer containing a primary amine in a side chain of the cationic polymer to thereby form reversible covalent bonds with amino groups. Moreover, the present invention aimed at further stabilization of micelles by polyion complex (PIC) formation. The object of the present invention is to stabilize the structure of micelles by covalent bonding and PIC formation and thereby enhance their blood retention.

Namely, the present invention is as follows.
[1] . . . . A pH-responsive carrier for nucleic acid delivery to cells or tissues, comprising a combination of a cationic polymer having a side chain containing a primary amine and a block copolymer represented by the following formula (1):

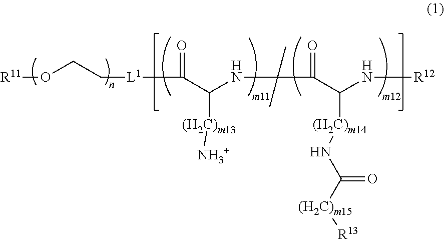

[wherein R¹¹ and R¹² each independently represent a hydrogen atom, or an optionally substituted linear or branched alkyl group containing 1 to 12 carbon atoms, or an azide, an amine, maleimide, a ligand or a labeling agent,
   R¹³ represents a compound represented by the following formula (I):

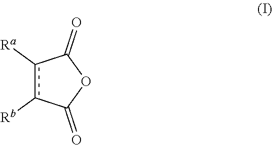

(wherein R^{a} and R^{b} each independently represent a hydrogen atom, or an optionally substituted alkyl group, an alkenyl group, a cycloalkyl group, an aryl group, an aralkyl group, an acyl group, a heterocyclic group, a heterocyclic alkyl group, a hydroxy group, an alkoxy group or an aryloxy group. Alternatively, R^{a} and R^{b} may be joined with each other to form an aromatic ring or a cycloalkyl ring together with the carbon atoms to which they are attached respectively. The bond between the carbon atoms to which R^{a} and R^{b} are attached respectively may be a single bond or a double bond),
   L¹ represents NH, CO, or a group represented by the following formula (11):

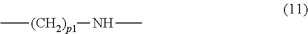

(wherein p1 represents an integer of 1 to 6), or
a group represented by the following formula (12):

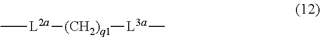

(wherein L^{2a} represents OCO, OCONH, NHCO, NHCOO, NHCONH, CONH or COO,
L^{3a} represents NH or CO, and q1 represents an integer of 1 to 6),
   m11 and m12 each independently represent an integer of 1 to 500 (provided that the sum of m11 and m12 represents an integer of 10 to 500), m13, m14 and m15 each independently represent an integer of 1 to 5, and n represents an integer of 1 to 500, and
   the symbol “/” means that (m11+m12) units of the respective monomer units shown on the left and right sides of this symbol may be in any sequence].
[2]. The carrier according to [1], wherein the cationic polymer having a side chain containing a primary amine is a polymer represented by the following formula (2), or a branched polyethyleneimine.

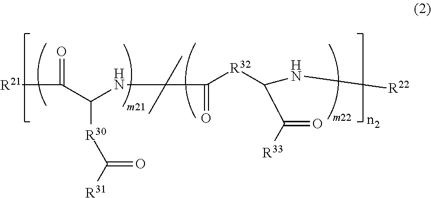

[wherein R²¹ and R²² each independently represent a hydrogen atom, or an optionally substituted linear or branched alkyl group containing 1 to 12 carbon atoms, or an azide, an amine, maleimide, a ligand or a labeling agent,
   R³⁰ represents (CH₂)ₘ₂₃ (m23 represents an integer of 1 to 5),
   R³² represents methylene group or ethylene group,
   R³¹ and R³³ each independently represent general formulae (41) or (42) below:

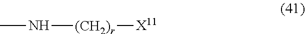

(wherein, X¹¹ represents an amine compound residue derived from primary amine compound, and r represents an integer of 0-5)

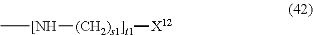

(wherein, X¹² is synonymous with X¹¹, and s1 and t1, independently from each other and independently between [NH—(CH₂)ₛ₁] units, represent integers of 1-5 and 2-5, respectively),
   m21 and m22 each independently represent an integer of 1 to 500 (provided that the sum of m21 and m22 represents an integer of 10 to 500),
   the symbol “/” means that (m21+m22) units of the respective monomer units shown on the left and right sides of this symbol may be in any sequence].
[3] The carrier according to [2], wherein R³¹ and R³³ each independently represent following group.

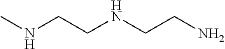

[4]. The carrier according to [1], wherein the compound represented by formula (I) is at least one of compounds represented by the following formulae (Ia) to (Ig).

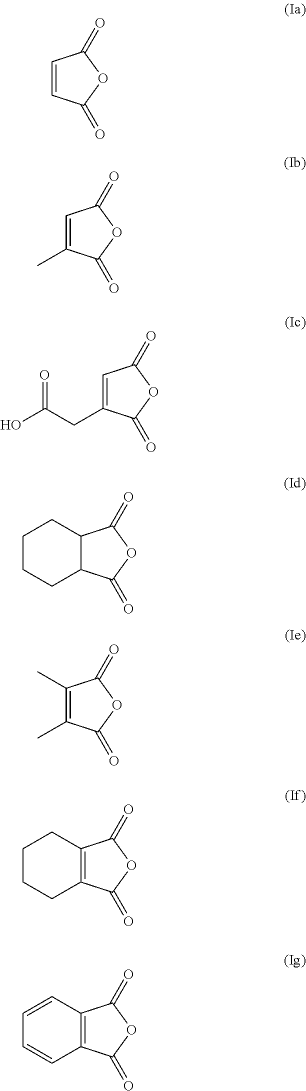

[5]. The carrier according to [4], wherein the compound represented by formula (I) is a compound represented by the following formula (Ia) or (Ib).

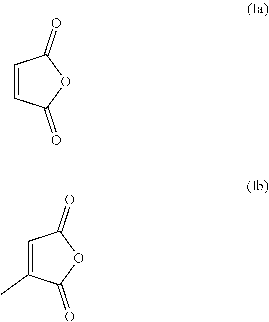

[6]. The carrier according to [1], wherein the block copolymer represented by formula 1 is a block copolymer represented by the following formula (3).

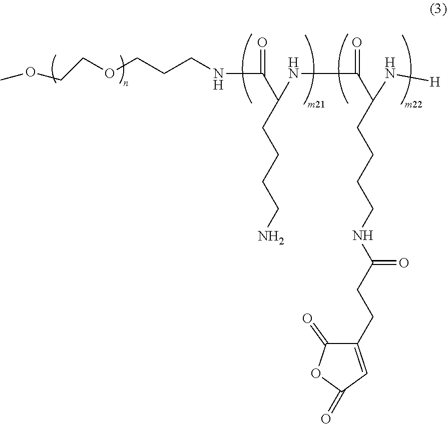

[7] A polyion complex comprising the carrier according to any one of [1] to [6], and nucleic acids.
[8]. The polyion complex according to [7], wherein the cationic polymer having a side chain containing a primary amine is covalently bonded to the block copolymer represented by the formula (1).
[9]. The polyion complex according to [8], wherein the covalent bond is cleaved in a pH-dependent manner.
[10]. A nucleic acid-delivery kit comprising the polyion complex according to [7] for use in nucleic acid delivery to target cells or tissues.
[11]. A nucleic acid-delivery device comprising the polyion complex according to [7] for use in nucleic acids delivery to target cells or tissues.

The present invention has enabled clinical applications of nucleic acids therapeutics. For example, siRNA can suppress expression of a disease-related gene in vivo, and mRNA can sustainably and safely produce therapeutic proteins. Moreover, the present invention can significantly suppress enzymatic degradation of RNA and augment transfection efficiency of RNA.

### BRIEF DESCRIPTION OF THE DRAWINGS

FIG. 1. a) 1H-NMR of PEG-pLL (polymer concentration: 10 mg/mL, solvent: D₂O, and temperature: 25° C.). b) GPC of PEG-pLL (polymer concentration: 1 mg/mL, solvent: PBS, and temperature: 25° C.).

FIG. 2. a) 1H-NMR of PEG-pLL-CAA (polymer concentration: 10 mg/mL, solvent: DMSO-d⁶, and temperature: 25° C.). b) GPC of PEG-p(LL-CAA) (polymer concentration: 1 mg/mL, solvent: PBS, and temperature: 25° C.).

FIG. 3. ¹H-NMR of Homo-P(Asp-det) (polymer concentration: 10 mg/mL, solvent: D₂O, and temperature: 25° C.).

FIG. 4. Micelle formation and characterization a) mRNA loaded micelles formation based on electrostatic interaction and covalent interaction. b) Size distribution by volume of mRNA loaded micelles. c) Normalized diffusion coefficient of micelles at buffered solution (10 mM HEPES with 150 mM NaCl) with different pHs. All results are expressed as the mean±s.d. (n=10).

FIG. 5. mRNA polyplex encapsulated PEG-p (LL-CAA) micelle formation. Homo-polyplex were first prepared by mixing mRNA and Homo-P(Asp-det) at N/P ratio of 1,2, 3, 6, 10 and 16, PEG-p (LL-CAA) solution (0.5 mg/mL) was gradually added to mRNA polyplex solution. a) Z-average diameter b) PDI c) derived count rate of the micelles determined by DLS measurement.

FIG. 6. mRNA polyplex encapsulated PEG-p (LL-CAA) micelle formation. Homo-polyplex were first prepared by mixing mRNA and Homo-P(Asp-det) at N/P ratio of 2, 6, 10 and 16, PEG-p (LL-CAA) solution (0.5 mg/mL) was gradually added to mRNA polyplex solution. Zeta potential of the micelles determined by DLS measurement.

FIG. 7. Stability of micelle a) Diffusion coefficient of micelles after incubation with sodium dextran sulfate at different S/P([sulfate in dextran sulfate]/[phosphate in mRNA]) ratios for 1 h. The upper dashed line represents the diffusion coefficient of naked mRNA. Data were expressed as the mean #s.d. (n=10). b) The remaining mRNA after incubating micelles with serum at 37° C. for 15 mins. The mRNA amount was measured by qRT-PCR. Data were expressed as the mean±s.d. (n=3), p values were calculated by the two-tailed student's t-test.

FIG. 8. In vitro performance of micelles. Fluorescence in cultured CT 26 cells with a) naked Cy5-labeled mRNA, b) PEG-pLL/m and c) PEG-pLL/m loading Cy5-labeled mRNA for 6 h incubation in medium, observed by confocal laser scanning microscopy (CLSM). Scale bars, 20 μm. d) Cellular uptake efficiency quantified from the mean fluorescence intensity of the pixels corresponding to Cy5 (n=30 cells). e) Efficiency of gluc protein expression in CT 26 cells after 24 h incubation in CT 26 cells. Data were expressed as the mean±s.d. (n=3). Statistical significance was calculated by the two-tailed student's t-test.

FIG. 9. In vitro transfection of mRNA polyplex encapsulated PEG-p (LL-CAA) micelle in CT 26 cells, HCT 116 cells, B16F10 cells, RAW246.7 cells, DC2.4 cells, HEK293 cells and BXPC3 cells after 24 h incubation. Data were expressed as the mean±s.d. (n=3).

FIG. 10. In vivo transfection of micelles. a) Representative bioluminescence images following in vivo delivery of fluc mRNA in CT 26 tumor-bearing mice. Mice were intratumorally injected with naked fluc mRNA, fluc mRNA loaded PEG-pLL/m, fluc mRNA PEG-pLL (CAA)/m and PEI fluc mRNA polyplexes (5 μg mRNA per mouse, n=3), and imaged at 6 h, 9 h, and 24 h post-injection. Quantification of luminescent signals from IVIS images at b) 6 h, c) 9 h, and d) 24 h post-injection. Data were expressed as the mean±s.d. (n=3). Statistical significance was calculated by the two-tailed student's t-test. e) Quantification analysis luminescent signals at indicated time points.

FIG. 11. Gel electrophoresis of PEI-doped mRNA-loaded micelles. Free mRNA was not detectable, indicating the encapsulation of mRNA in the micelles.

FIG. 12. TEM images of micelles after staining with 1% uranyl acetate. The size distribution was analyzed by the histogram in the lower panel.

FIG. 13. Efficiency of Gluc protein expression in CT26 and RAW 264.7 cells after 24 h incubation. Data were expressed as mean #s.d. (n=4).

### DESCRIPTION OF EMBODIMENTS

Although therapeutic nucleic acids are expected to be promising in the treatment of intractable diseases, their systemic administration involves various problems including instability, short half-life, and non-specific immune reactions, etc. Thus, a nucleic acid delivery approach using stimuli-responsive nanocarriers may be an effective strategy to enhance nucleic acid activity in target tissues in a tissue selective manner. In the present invention, there have been developed polymeric micelles having the ability to form a polyion complex between nucleic acids and block copolymer and thereby encapsulate the nucleic acids through covalent bonding cleavable under given pH conditions, with the aim of releasing the loaded nucleic acids in a pH-dependent manner.

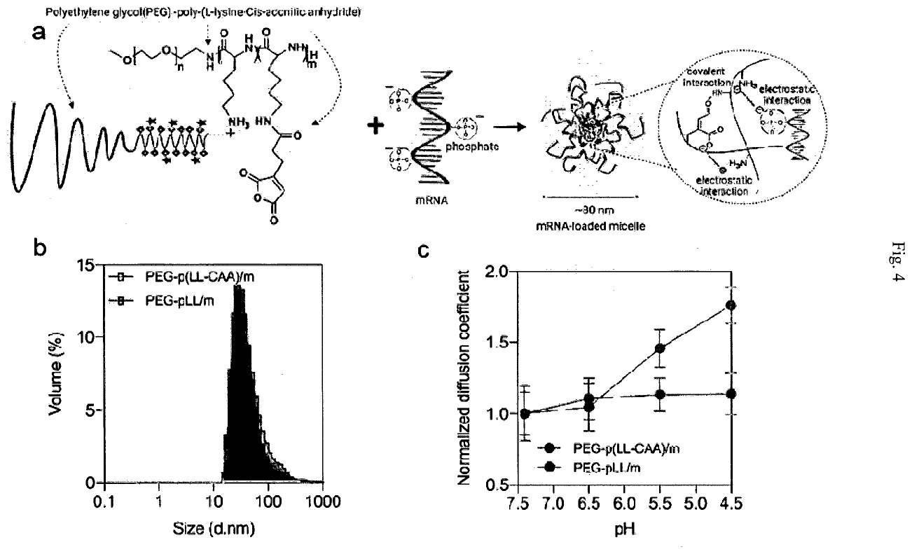

In the present invention, a cationic polymer having a primary amine in its side chain was first mixed with a nucleic acid to prepare a polymer-nucleic acid complex (polyplex). In this complex, electrostatic bonding is formed between the nucleic acid and the cationic polymer. Subsequently, a block copolymer containing a pH-responsive maleic anhydride derivative is introduced into the complex to form a reversible covalent bond with the amino group of the cationic polymer, thereby increasing micelle stability, and efficient release of nucleic acids under acidic conditions. Schematic illustration explaining the micelle formation is shown in FIG. 4a.

The polymeric complex of the present invention is a nucleic acids-enclosing polymeric micellar complex (polyion complex: PIC), which comprises a particular type of cationic polymer and nucleic acids.

1. A pH-Responsive Carrier for Nucleic Acid Delivery

The pH-responsive carrier for nucleic acid delivery to cells or tissues comprises a combination of a cationic polymer having a side chain containing a primary amine and a block copolymer represented by the following formula (1):

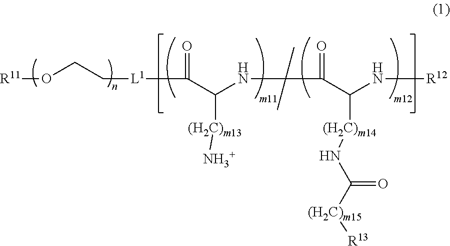

### (1) Cationic Polymer Having a Side Chain Containing a Primary Amine

A particular type of cationic polymer, which is a member constituting the PIC of the present invention, is a cationic polymer at least partially having a polycation moiety. Such a cationic polymer may be, for example, a block copolymer or graft polymer having a polycation moiety, without being limited thereto. Depending on the intended use of the PIC of the present invention, a preferred embodiment may be selected as appropriate.

The above PEG and polycation have no limitation on their structure (e.g., their degree of polymerization), and those of any structure may be selected. Above all, preferred as a polycation is a polypeptide having cationic groups in its side chains. As used herein, the term “cationic group” is intended to mean not only a group which is already cationic by being coordinated with hydrogen ions, but also a group which will be cationic when coordinated with hydrogen ions. Such cationic groups include all of the known ones.

More specifically, the cationic polymer having a side chain containing a primary amine is a polymer represented by the following formula (2), or a branched polyethyleneimine.

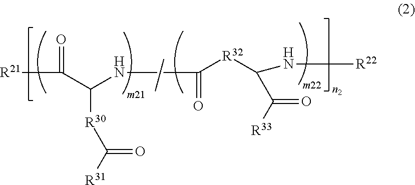

[wherein R²¹ and R²² each independently represent a hydrogen atom, or an optionally substituted linear or branched alkyl group containing 1 to 12 carbon atoms, or an azide, an amine, maleimide, a ligand or a labeling agent,
R³⁰ represents (CH₂) m23 (m23 represents integer of 1 to 5),
R³² represents methylene group or ethylene group,
R³¹ and R³³ each independently represent general formulae (41) or (42) below:

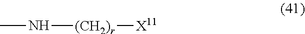

(wherein, X¹¹ represents an amine compound residue derived from primary, secondary or tertiary amine compound or quaternary ammonium salt, and r represents an integer of 0-5)

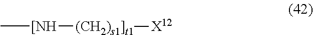

(wherein, X¹² is synonymous with X¹¹, and s1 and t1, independently from each other and independently between [NH—(CH₂) s1] units, represent integers of 1-5 and 2-5, respectively),
m21 and m22 each independently represent an integer of 1 to 500 (provided that the sum of m21 and m22 represents an integer of 10 to 500),
the symbol “/” means that (m21+m22) units of the respective monomer units shown on the left and right sides of this symbol may be in any sequence].

In the present invention, a structure of the branched polyethyleneimine is as follows:

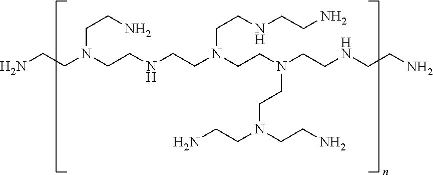

The cationic polymer having a side chain containing a primary amine can be prepared, for example, as shown in Example.

### (2) Block Copolymer Represented by the Formula (1)

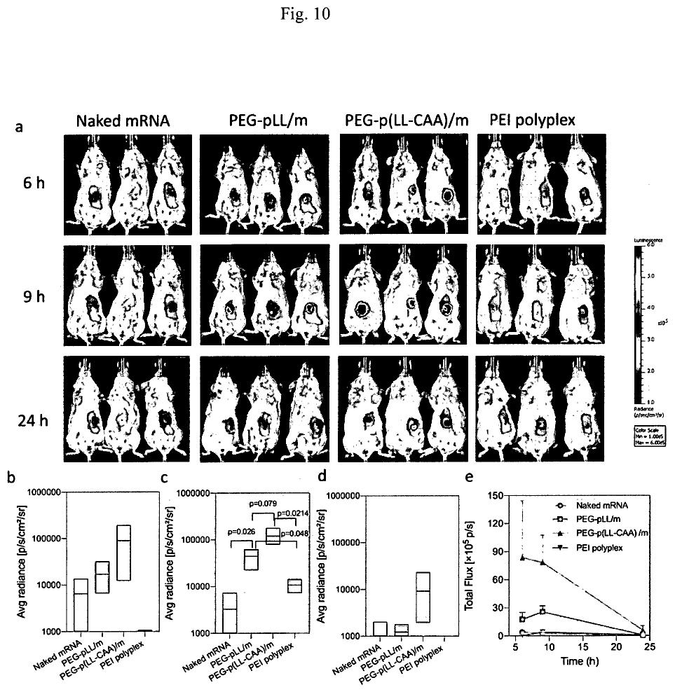

A cis-aconitic anhydride (CAA)-amide bond is stable at physiological pH (pH 7.4), but is cleaved at pH 6.5, i.e., at pathophysiological pH in tumors and inflammatory tissues. For this reason, CAA was selected as a pH-responsive functional group. In the present invention, a poly(ethylene glycol)-poly(L-lysine) block copolymer with CAA was used. In the Example, mRNA-enclosing micelles were used as a model to confirm micelle stability under physiological conditions, as well as micelle breakdown and functional mRNA release at pH 6.5. Further, PEG-pLL (CAA)/m were found to have an enhanced protein expression when compared to naked mRNA alone (FIG. 10). Thus, the above model indicated the usefulness of the system for in vivo delivery of therapeutic nucleic acids.

More specifically, the above particular type of cationic polymer may preferably be exemplified by a block copolymer represented by the following general formula (1).

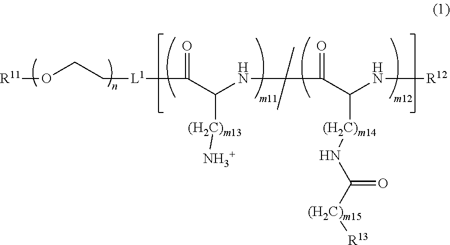

In the structural formula shown in general formula (1), the block moiety whose number of repeating units (degree of polymerization) is n corresponds to the PEG moiety, while the block moiety composed collectively of submoieties whose number of repeating units is m11 and m12, respectively (i.e., the moiety shown in brackets [ ] in general formula (1)) corresponds to the polycation moiety. Moreover, the symbol “/” appearing in the structural formula of the polycation moiety is intended to mean that the respective monomer units shown on the left and right sides of this symbol may be in any sequence. For example, when a block moiety composed of monomer units A and B is represented by [-(A)a-/-(B) b-], the symbol “/” means that a units of A and b units of B, i.e., (a+b) units in total of the respective monomer units may be linked at random in any sequence (provided that all the monomer units A and B are linked in a linear fashion).

In general formula (1), R¹¹ and R¹² each independently represent a hydrogen atom, or an optionally substituted linear or branched alkyl group containing 1 to 12 carbon atoms, or a functional group such as an azide, an amine, maleimide, a ligand or a labeling agent.

Examples of the above linear or branched alkyl group containing 1 to 12 carbon atoms include a methyl group, an ethyl group, a n-propyl group, an isopropyl group, a n-butyl group, a sec-butyl group, a tert-butyl group, a n-pentyl group, a n-hexyl group, a decyl group and an undecyl group, etc. Moreover, examples of substituents on the above alkyl group include an acetal-protected formyl group, a cyano group, a formyl group, a carboxyl group, an amino group, an alkoxycarbonyl group containing 1 to 6 carbon atoms, an acylamido group containing 2 to 7 carbon atoms, a siloxy group, a silylamino group, and a trialkylsiloxy group (each alkylsiloxy group independently contains 1 to 6 carbon atoms), etc.

A ligand molecule refers to a compound used with the aim of targeting a certain biomolecule, and examples include an antibody, an aptamer, a protein, an amino acid, a low molecular compound, a monomer of a biological macromolecule and so on. Examples of a labeling agent include, but are not limited to, fluorescent labeling agents such as a rare earth fluorescent labeling agent, coumarin, dimethylaminosulfonyl benzoxadiazole (DBD), dansyl, nitrobenzoxadiazole. (NBD), pyrene, fluorescein, a fluorescent protein and so on.

When the above substituent is an acetal-protected formyl group, this substituent can be converted into another substituent, i.e., a formyl group (or an aldehyde group; —CHO) upon hydrolysis under acidic mild conditions. Moreover, when the above substituent (particularly on R¹¹) is a formyl group or is a carboxyl group or an amino group, for example, an antibody or a fragment thereof or other functional or targeting proteins may be linked via these groups.

In general formula (1), R¹³ represents a compound represented by the following general formula (I).

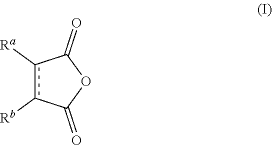

In the above formula (I), R^{a} and R^{b} each independently represent a hydrogen atom, or an optionally substituted alkyl group, an alkenyl group, a cycloalkyl group, an aryl group, an aralkyl group, an acyl group, a heterocyclic group, a heterocyclic alkyl group, a hydroxy group, an alkoxy group or an aryloxy group. Alternatively, R^{a} and R^{b} may be joined to form an aromatic ring or a cycloalkyl ring together with the carbon atoms to which they are attached respectively. Moreover, in formula (I), the bond between the carbon atoms to which R^{a} and R^{b} are attached respectively may be a single bond or a double bond, i.e., is not limited in any way. In formula (I), to express these two bonding modes collectively, the bond between these carbon atoms is represented by a combination of one solid line and one broken line.

L¹ represents NH, CO, a group represented by the following general formula (11):

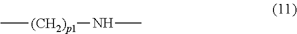

(wherein p1 represents an integer of 1 to 6), or
a group represented by the following general formula (12):

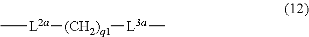

(wherein L^{2a} represents OCO, OCONH, NHCO, NHCOO, NHCONH, CONH or COO, L^{3a} represents NH or CO, and q1 represents an integer of 1 to 6).

In the above formula (1), m11 and m12 each independently represent an integer of 1 to 500 (provided that the sum of m11 and m12 represents an integer of 10 to 500), and m13, m14 and m15 each independently represent an integer of 1 to 5. In the above formula (1), n represents the number of repeating units (degree of polymerization) in the PEG moiety, and more specifically represents an integer of 1 to 500 (preferably 100 to 400, more preferably 200 to 300).

The molecular weight (Mn) of the cationic polymer represented by general formula (1) is not limited in any way, but it is preferably 23,000 to 45,000, and more preferably 28,000 to 34,000. With regard to the individual block moieties, the PEG moiety has a molecular weight (Mw) of preferably 8,000 to 15,000, and more preferably 10,000 to 12,000, while the polycation moiety as a whole has a molecular weight (Mn) of preferably 15,000 to 30,000, and more preferably 18,000 to 22,000.

The cationic polymer represented by general formula (1) may be prepared in any manner. For example, a segment comprising R¹¹ and the block moiety of PEG chain (PEG segment) is synthesized in advance, and given monomers are sequentially polymerized to one end (opposite to R¹¹) of this PEG segment, optionally followed by substituting or converting each side chain to contain a cationic group, or alternatively, the above PEG segment and a block moiety containing cationic groups in its side chains are synthesized in advance, which are then liked to each other. Procedures and conditions for each reaction in these preparation processes may be selected or determined as appropriate in consideration of standard processes.

In one embodiment of the present invention, the compound represented by formula (I) is at least one of compounds represented by the following formulae (Ia) to (Ig).

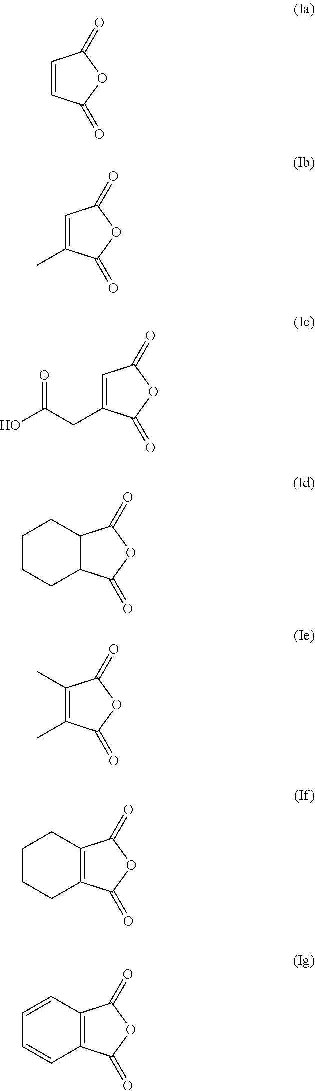

In a preferred embodiment of the present invention, the compound represented by formula. (I) is a compound represented by the following formula (Ia) or (Ib).

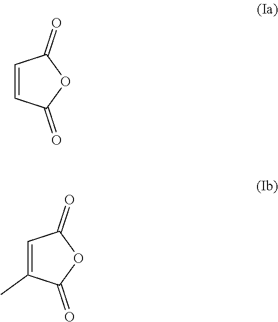

In formula (I), possible substituents may be saturated or unsaturated non-cyclic or cyclic hydrocarbon groups. In the case of non-cyclic hydrocarbon groups, they may be either linear or branched. Examples of such hydrocarbon groups include a C₁-C₂₀ alkyl group, a C₂-C₂₀ alkenyl group, a C₄-C₂₀ cycloalkyl group, a C₆-C₁₈ aryl group, a C₆-C₂₀ aralkyl group, a C₁-C₂₀ alkoxy group, and a C₆-C₁₈ aryloxy group.

The above compound represented by formula (I) is bonded (covalently bonded) to an amino group in a cationic polymer having a side chain containing a primary amine to form a structure as represented by the following formula (I′).

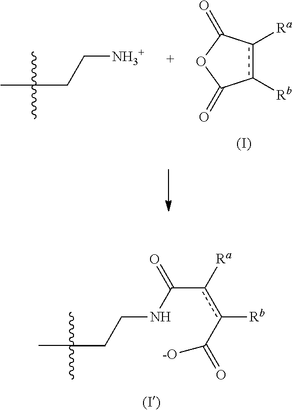

As to the above bonding, for example, when the above compound represented by formula (I) is a compound represented by formula (Ib) or (Ic) shown above, the above structure represented by formula (I′) formed after the bonding is as shown below.

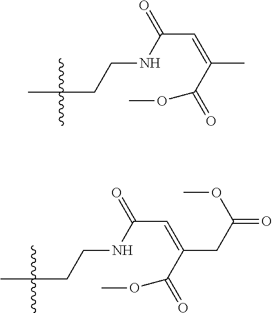 [SMILES: *CCNC(=O)/C=C(/C)C(=O)OC ; *CCNC(=O)/C=C(/CC(=O)OC)C(=O)OC]

In a further embodiment of the present invention, the block copolymer represented by formula 1 is represented by the following formula 2.

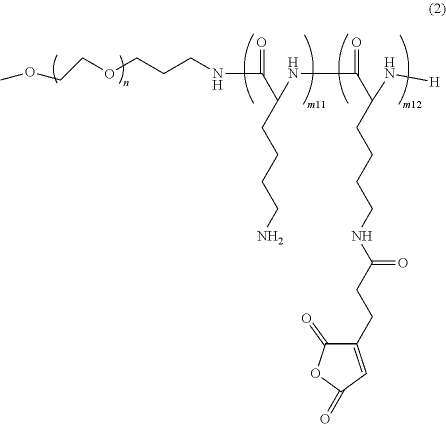

### (3) Nucleic Acids

In one aspect, nucleic acids encapsulated in polyion complex micelles of the present invention as a component of the core moiety are DNA or RNA. Examples of RNA include mRNA, siRNA (small interfering RNA), antisense nucleic acids (antisense RNA), an aptamer (RNA aptamer), self-replicating RNA, miRNA (microRNA) and lncRNA (long non-coding RNA). Examples of DNA include antisense nucleic acids (antisense DNA), an aptamer (DNA aptamer), pDNA (plasmid DNA) and MCDNA (minicircle DNA).

Any siRNA can be used as long as it can suppress the expression of the targeted gene through RNA interference (RNAi), where examples of the target gene favorably include, but not limited to, a cancer (tumor) gene, an anti-apoptosis gene, a cell cycle-related gene and a growth signal gene. Moreover, the base pair of siRNA is generally not limited as long as it is less than 30 base pairs (for example, 19-21 base pairs).

Since nucleic acids such as siRNA are anionic molecules, it can interact (assemble) with the cationic polymer having a side chain containing a primary amine via electrostatic interaction.

### (4) Polyion Complex (PIC)

The PIC of the present invention can be regarded as a core-shell type micellar complex in such a state where the nucleic acids and a part (polycation moiety) of the above cationic polymer form a core region through their electrostatic interaction, and other parts (including the PEG moiety) in the cationic polymer form a shell region around the core region.

Polyion complex micelles of the present invention are supramolecular assemblies that can be obtained by mixing nucleic acids and polymers of the present invention in buffer, which is also called a polyion complex (PIC) or a polyion complex-type polymeric micelle (PIC micelle).

The polymer complex of the present invention can be prepared by mixing, for example, nucleic acids and the polymer compound in an arbitrary buffer. If the polymer utilized to form polyion complex micelles of the present invention is a cationic polymer having a side chain containing a primary amine, the nucleic acids and the polycation can assembly via electrostatic interaction to form a polyplex structure. Subsequently, a block copolymer containing a pH-responsive maleic anhydride derivative represented by formula (1) is introduced into the complex to form a reversible covalent bond with the amino group of the cationic polymer.

The PIC of the present invention may be readily prepared, for example, by mixing the nucleic acids and the cationic polymer in any buffer (e.g., Tris buffer). The mixing ratio between the cationic polymer and the nucleic acids is not limited in any way. However, in the present invention, for example, the ratio between the total number (N) of cationic groups (e.g., amino groups) in the block copolymer and the total number (C) of phosphate_groups in the nucleic acids (N/C ratio) may be set to 0.1 to 200, particularly 0.5 to 100, and more particularly 1 to 50. If the N/C ratio is within the above range, it is preferred in that free molecules of the cationic polymer can be reduced. It should be noted that the above cationic groups (N) are intended to mean groups capable of forming ionic bonds through electrostatic interaction with phosphate_groups in the nucleic acids to be enclosed within the micelle.

The PIC of the present invention is of any size. For example, its particle size is preferably 5 to 200 nm, and more preferably 10 to 100 nm, as measured by dynamic light scattering (DLS).

Upon introduction into cells or tissues, the PIC of the present invention will release the nucleic acids enclosed therein. In this case, the above compound represented by formula (I) is dissociated (cleaved) from the nucleic acids in response to a change in the pH environment within the cytoplasm (which is changed to a weakly acidic environment (e.g., around pH 5.5)). As a result, the charge (overall charge) of the nucleic acids as a whole returns to the original charge (overall charge) inherent to the nucleic acids, so that the nucleic acids can be present within the recipient cells in a state where its structure and activity, etc. are regenerated.

### 2. Kit for a Nucleic Acid Delivery Device

A kit for a nucleic acid delivery device of the present invention is characterized by comprising a polymer compound of the present invention. This kit can preferably be used, for example, for gene therapy using RNAi for a target cell and protein therapy using mRNA.

In the kit of the present invention, the preserved form of the polymer is not limited, and it can be selected from a solution form, a powdery form or the like considering its stability (storability), ease of use, and else.

In addition to the above-described polymer compound, the kit of the present invention may include other components. Examples of other components include nucleic acids to transfect into the cells, buffers used for dissolving, diluting and the like, protein and an instruction (instruction manual), which may suitably be selected according to the purpose of use and the type of polymer.

The kit of the present invention is used for preparing polyion complex (PIC) micelles which encapsulate nucleic acids (for example, mRNA or siRNA) in the core to transfect into targeted cells. The prepared PIC holds advantages as a device for delivering nucleic acids to targeted cells.

### 3. Device for Delivering Nucleic Acids

The present invention can provide a nucleic acid delivery device comprising the above-described polyion complex. The delivery device of the present invention is capable of stabilizing nucleic acids which had been difficult to stably deliver to targeted cells by enhancing the resistance against enzyme degradation.

The delivery device of the present invention can be applied to various animals including, but not limited to, humans, mice, rats, rabbits, pigs, dogs, cats and the like. A parenteral method such as intravenous injection is usually employed for administering the device to a subject animal, where various conditions including the amount, number and period of administration are suitably determined according to the type and the state of the subject animal.

The delivery device of the present invention can be used for a treatment where desired nucleic acids are transfected into cells in pathological sites (gene therapy). Hence, the present invention can also provide a pharmaceutical composition comprising the above-described polyion complex for treating various diseases, gene therapeutics for various diseases which comprises pharmaceutical composition, and a method for treating various diseases which comprises use of PIC (particularly, gene therapy).

Methods and conditions are the same as described above. Moreover, examples of the various diseases include, but not limited to, cancers (for example, lung cancer, pancreas cancer, brain tumor, liver cancer, breast cancer, colorectal cancer, neuroblastoma and bladder cancer), cardiovascular diseases, musculoskeletal diseases and central nervous system diseases.

The above-described pharmaceutical composition can be formulated by an ordinary method by suitably selecting and using a diluent, a filler, a bulking agent, a binder, a wetting agent, a disintegrant, a lubricant, a surfactant, a dispersant, a buffer, a preservative, a solubilizing aid, an antiseptic, a flavoring agent, a soothing agent, a stabilizer, a tonicity adjusting agent and the like which are generally used for drug production. In addition, an intravenously injectable agent (including intravenous drip) is usually employed as the form of the pharmaceutical composition. For example, the pharmaceutical composition may be provided in a single-dose ampoule or in a multiple-dose container. cells.

### EXAMPLES

The present invention will be further described in more detail by way of the following illustrative examples, which are not intended to limit the scope of the invention.

### Example 1

### 1. Materials and Methods

### 1.1. Materials

α-Methoxy-ω-amino poly(ethylene glycol) (MeO-PEG-NH₂) (M_{w}=12 kDa) was purchased from NOF CORPORATION (Tokyo, Japan). ε-Trifluoroacetyl-L-lysine N-carboxyanhydride (Lys-(TFA)-NCA) was purchased from Chuo Kaseihin Co. Inc. (Tokyo, Japan). N,N-Dimethylformamide (DMF) (purity>99.5%), methanol (purity>99.5%) were purchased from Fujifilm Wako Pure Chemical, Co., Inc., (Tokyo, Japan). Diethyl ether (purity>95%), cis-aconitic anhydride (purity>95%), dextran sulfate (Mᵣ˜40,000), and 4-(2-Hydroxyethyl)-1-piperazineethanesulfonic acid (HEPES) (1.0 M), Fetal bovine serum (FBS), Penicillin-Streptomycin, and RPMI-1640 Medium were purchased from Sigma-Aldrich (St. Louis, Missouri, USA). Oxalyl Chloride (purity>98%) and anhydrous dichloromethane (purity>98%) were purchased from Tokyo Chemical Industry Co., Ltd. (Tokyo, Japan).

### 1.2. Polymer Synthesis

PEG-pLL block copolymer was prepared as previously reported [8]. poly(ethylene glycol)-b-poly[L-lysine (TFA)] (PEG-pLL (TFA)) was firstly synthesized by ring opening polymerization (ROP) taking MeO-PEG-NH₂ (Mw=12000 g mol-1) as initiator. In brief, MeO-PEG-NH₂ (1 g, 0.083 mmol) and Lys (TFA)-NCA (1 g, 3.75 mmol) were separately dissolved in 10 mL anhydrous DMF. Two solutions were then mixed under Ar flow and allowed for reaction in 35° C. water bath for 48 h. The mixture was precipitated against diethyl ether to get the PEG-pLL (TFA). Deprotection of the TFA group was completed by dissolving the collected PEG-pLL (TFA) in methanol with 1M NaOH and kept reacting in 35° C. water bath for 12 h. The mixture was purified by dialysis against 0.01 M HCl and pure water (molecular weight cut-off (MWCO): 6,000-8,000 Da), then lyophilized to get the poly(ethylene glycol)-b-poly(L-lysine) (PEG-pLL). The degree of polymerization (DP) of the lysine groups was determined by 1H-NMR (400 MHZ, JEOL ECS-400, JEOL, Tokyo, Japan) in D₂O and the polydispersity of the polymer was tested by the aqueous GPC (Extrema 4500Model, JASCO) (eluent: 10 mM PBS, pH 7.4; temperature: 25° C.; flow rate: 0.75 mL min-1; detector: UV 220 nm).

PEG-pLL (CAA) block copolymer were synthesized by conjugating further cis-aconitic anhydride (CAA) molecules with the amino groups in lysine block of the PEG-pLL via condensation reaction between acid chloride and amine. Briefly, CAA (153 mg, 1 mmol) was reacted with Oxalyl chloride (2 mL, 2.5 g, 20 mmol) at 25° C. for one overnight to prepare the acid chloride of CAA (CAA-CI). The CAA-Cl was purified by vacuum evaporation to totally remove the exceeded oxalyl chloride, the product was collected as an oily liquid. PEG-pLL (200 mg, 0.011 mmol) was then dissolved in 20 mL anhydrous anhydrous dichloromethane and then reacted with the prepared CAA-Cl at 25° C. for one overnight. The final product, PEG-pLL (CAA), was obtained by precipitating the mixture against diethyl ether. The number of CAA units in PEG-pLL was confirmed by 1H-NMR in DMSO-d6 at 80° C. and the polydispersity of the polymer was tested by the aqueous GPC (eluent: 10 mM PBS, pH 3.0; temperature: 25° C.; flow rate: 0.75 mL min-1; detector: UV 220 nm).

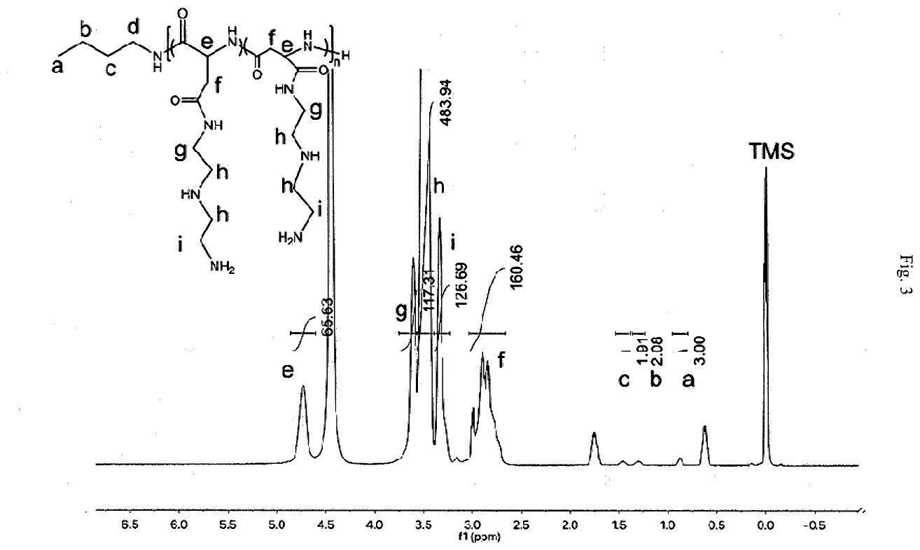

To prepare Homo-P(Asp-det), Homo-PBLA was synthesised via an ROP reaction of the BLA-NCA initiated by n-butylamine. n-Butylamine (50.0 μL, 0.506 mmol) and BLA-NCA (10.0 g, 40.1 mmol) were separately dissolved in 10 mL anhydrous DMF. Two solutions were then mixed under Ar flow and allowed for reaction in at 35° C. for 48 h. The mixture was precipitated against diethyl ether to get the Homo-PBLA powder. Homo-pAsp (DET) was then synthesised by aminolysis of Homo-PBLA. Homo-PBLA (120 mg, 0.01 mmol) was dissolved in 10 mL anhydrous DMF. Diethylenetriamine (DET) (2.5 mL, 25 mmol) was added to the Homo-PBLA solution. The mixture was reacted at 0° C. for 1 h, then the reaction was stopped by the dropwise addition of ice-cold 5 M HCl(aq) equivalent to the amine groups fed in the solution. The neutralised solution was firstly dialysed against 0.01 M HCl (MWCO: 6,000-8,000 Da), then change to dialysis against pure water. The purified solution was lyophilised to get Homo-pAsp (DET) as a white powder. The final product was characterised by 1H-NMR in D₂O for confirming the conjugation of DET groups (FIG. 3).

### 1.3. In Vitro Transcribed mRNA

Plasmid RNA temples for preparing Gaussian luciferase (gluc) and Firefly luciferase (fLuc) mRNA were prepared by inserting corresponding protein coding sequences having 120 bp poly A/T sequence into pSP73 vector (Promega, Madison, Wisconsin, USA). Linearized gluc and fLuc plasmids were used as temples for in vitro transcription using mMESSAGE mMACHINE™ T7 Ultra Kit (Thermo Fisher Scientific, Waltham, Massachusetts, USA) to produce gluc and fluc mRNA. Obtained mRNA was then purified by RNeasy Mini Kit (Qiagen, Hilden, Germany). The mRNA concentration was determined by measuring the absorbance at 260 nm using NanoDrop 3300 spectrophotometer (Thermo Fisher Scientific).

### 1.4. Micelle Preparation

To prepare mRNA loading PEG-pLL/m, synthesized mRNA was dissolved in 10 mM HEPES buffer at the concentration 25 ng/μL. PEG-pLL block copolymer was also dissolved in 10 mM HEPES buffer, the concentration of PEG-pLL was adjusted to match the molar ratio of [amine groups in polymer (N)]/[phosphate groups in mRNA (P)]. Micelles were then kept at 4° C. for 1 h before use.

To prepare mRNA loading PEG-pLL (CAA)/m, synthesized mRNA and PEG-pLL (CAA) polymer were dissolved in pH 8.5 and pH 3.5 10 mM HEPES buffer respectively. PEG-pLL (CAA) solution (0.5 mg/mL) was then gradually added to mRNA solution (25 μg/mL) at N/P ratio of 4. The volume ratio of polymer solution to mRNA solution was controlled at 1:1. Finally, pH of micelle solution was then adjusted to 7.4 by adding pH 8.5 10 mM HEPES and left stirring for 2 h before use.

To prepare mRNA polyplex loading PEG-pLL (CAA)/m, homo-polyplex were first prepared by mixing mRNA and Homo-P(Asp-det) at N/P ratio of 2, 6, 10 and 16, the final mRNA concentration was adjusted to 25 μg/mL. Polyplex were then kept at 4° C. for 30 mins, then PEG-pLL (CAA) solution (0.5 mg/mL) was gradually added to mRNA solution. The volume ratio of polymer solution to mRNA solution was controlled at 1:1. Finally, pH of micelle solution was then adjusted to 7.4 by adding pH 8.5 10 mM HEPES and left stirring for 2 h before use.

### 1.5. Micelle Characterization

The Z-average diameter of micelle (25 ng/μL of mRNA) was measured by dynamic light scattering (DLS) using Zetasizer Nano ZS (Malvern Instruments Ltd, UK). mRNA loading was also confirmed by fluorescence correlation spectroscopy (FCS) measurement. Firstly, mRNA was labeled with Cy5 was using Label IT Tracker Intracellular Nucleic Acid Localization Kit (Mirus Bio Corporation, Madison, WI, USA). Using this Cy5-labeled mRNA, PEG-pLL/m and PEG-PGBA/m were prepared as described above. Micelle solution was then diluted with down to 2 mM Cy5-labeled mRNA with 10 mM HEPES buffer (pH 7.4). Diluted micelle solution (200 μL) was then performed FCS measurement via He—Ne laser (633 nm) scanning. Herein, Alexa Fluor 647 dye was selected as a standard, the diffusion coefficient of Cy5-labeled mRNA was obtained by comparing the comparing their diffusion time with that of Alexa Fluor 647. Moreover, the obtained count per molecule was analyzed to calculate the association mRNA number per micelle according to the following equation:

Association number=Count per molecule (Micelle)/Count per molecule (Naked mRNA)

### 1.6. Micelle pH Sensitivity

To examine the pH sensitivity of the PEG-pLL (CAA)/m, Cy5-labeled gluc mRNA, micelles were prepared and incubating with 10 mM HEPES buffer with 150 mM NaCl at pH 7.4, 6.5, 5.5 and 4.5 at room temperature for 1 h. Samples were then analyzed by FCS to track the micelle dissociation.

### 1.7. Micelle Stability Against Polyanion

Micelle stability against counter polyanion exchange was estimated by mixing micelle with sodium dextran sulfate and followed by FCS measurement. Briefly, Cy5-labeled mRNA micelle solutions containing 200 ng Cy5-labeled mRNA were mixed with sodium dextran sulfate at different S/P([sulfate in dextran sulfate]/[phosphate in mRNA]) ratios, the resulting solution was then kept at room temperature for 1 hour, the diffusion coefficient of Cy5-labeled mRNA were obtained by FCS measurement as mentioned above.

### 1.8. FBS Stability

To test micelle stability against nuclease, micelles loaded with gluc mRNA were incubated in 10% FBS (final mRNA concentration was adjusted to 6.25 ng/μL) for 15 min at 37° C. The resulting mRNA containing solution were cleaned by RNeasy Mini Kit, then reverse-transcribed with the ReverTra Ace qPCR RT Master Mix kit. Finally, qRT-PCR analysis was eventually performed by the 7500 Fast Real-Time PCR Instrument (Applied Biosystems, USA) using a primer pair for gluc mRNA (Forward; TGAGATTCCT GGGTTCAAGG, and Reverse; GTCAGAACACTGCACGTTGG)

### 1.9. In Vitro Uptake

Micelle uptake by cells was measured by LSM 780 confocal laser scanning microscope. CT 26 cells (10,000 cells) on 8-well chambered borosilicate cover glass (Lab Tek) and incubated in RPMI containing 10% FBS and 1% penicillin/streptomycin with 5% CO₂ at 37° C. After 24 h, Cy5-labeled gluc mRNA and mRNA encapsulated micelles (700 ng mRNA per well, relative fluorescence intensity: 400 [RFU]) were applied to CT 26 cells. After another 6 h, cells were washed with PBS for three times, cell nucleus was stained with 1% Hoechst33342 solution for 5 mins before LSM imaging.

### 1.10. In Vitro Cellular Transfection

CT 26 cells were cultured in RPMI containing 10% FBS and 1% penicillin/streptomycin with 5% CO₂ at 37° C. To evaluate gluc expression efficiency, cells were seeded onto 96-well plates at the density of 50,000 cells per well. After 24 h incubation, 500 ng of gluc mRNA, PEG-pLL/m and PEG-pLL (CAA)/m containing 250 ng of gluc mRNA was added. mRNAs were also complexed with in vivo-jetPEI (Polyplus-transfection, Illkirch-Graffenstaden, France) at 1.2 μl PEI/μg of mRNA and transfected into cells according to manufacturer's instructions. After another 24 hours, 50 μL culture medium was collected for luciferase assay by Renilla Luciferase Assay system (Promega, Madison, WI, USA) and GloMax 96 Microplate Luminometer (Promega, Madison, WI, USA).

### 1.11. In Vivo Transfection

To prepare CT 26 tumor model, 1×10⁶ CT 26 cells were inoculated into the flanks of female balb/c mice provided by Charles River Laboratories Japan, Inc. After about 2 weeks, palpable tumors were observed. Micelles and PEI polyplex were prepared as described above using 5 μg Firefly luciferase (fluc) mRNA per mouse. Mice were then randomized into four groups and received intratumoral injections with naked mRNA, PEG-pLL/m, PEG-pLL (CAA)/m and PEI polyplex. At 1 h, 9 h, and 24 h post-injection, mice were intra-peritoneally injected with 200 μL 50 mg/mL luciferin solution, fluc expression was imaged after 10 mins using IVIS Spectrum imaging system (SP-BFM-T1, PerkinElmer, Waltham, MA, USA),

### 1.12. Statistical Analysis

The results are presented as mean±standard deviation (s.d.). Groups were compared by performing two-tailed student's t-test in Graph Pad Prism 8.

### 2. Results and Discussion

### 3.1 Synthesis and Characterization of Block Copolymer

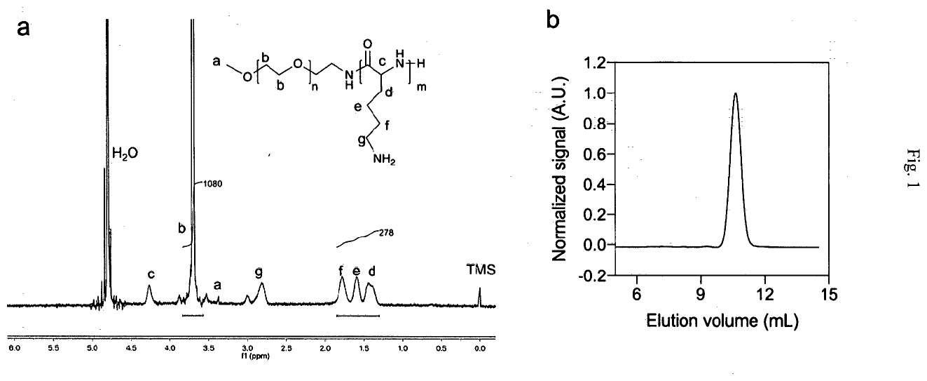

PEG-pLL (TFA) block copolymer was successfully synthesized from ROP of Lys (TFA)-NCA, taking the terminal amine of MeO-PEG-NH₂ as initiator as described in previous paper. The trifluoroacetyl groups were then cleaved by alkaline hydrolysis to get PEG-pLL, final product was characterized by 1H-NMR analysis (D₂O; 25° C.). The DP of the lysine groups was determined to be 46 units from ¹H-NMR by comparing the characteristic peaks of —O—CH₂—CH₂—O— (δ=3.57-3.84 ppm) in PEG block with the peaks of —CH₂—CH₂—CH₂— (δ=1.30-1.80 ppm) in PLL side chain and narrow molecular weight distribution was observed in the aqueous phase GPC (FIG. 1).

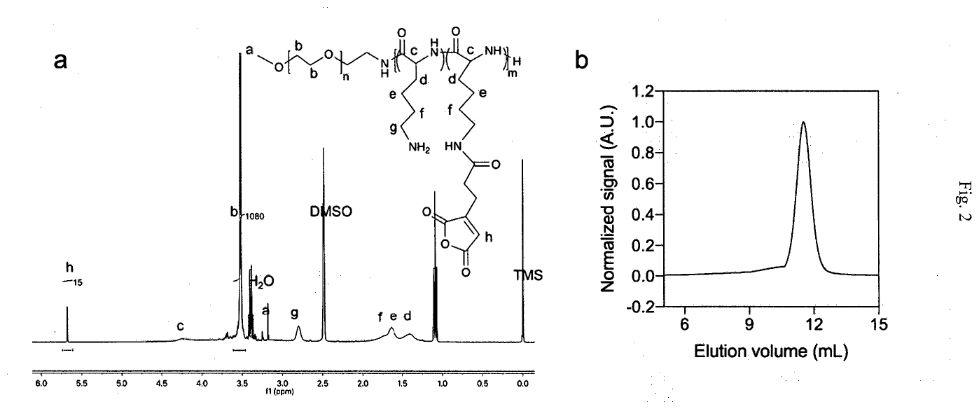

The resulting PEG-pLL block copolymer was then modified with CAA to produce PEG-pLL (CAA) by reacting CAA-CI with lysine groups. Introduced CAA units were determined to be 15 units from ¹H-NMR by comparing the peaks of —O—CH₂—CH₂—O— (δ=3.46-3.61 ppm) in PEG block with the characteristic peak of —CH— (δ=5.61-5.74 ppm) in CAA (FIG. 2a). Also, aqueous phase GPC result of the PEG-pLL (CAA) indicated polymer has narrow molecular weight distribution (FIG. 2b).

### 3.2 Micelle Formation and Characterization

PEG-pLL mRNA PIC micelles were assembled by mixing PEG-PLL with gLuc mRNA in 10 mm HEPES buffer. Based on our previous result, stable PEG-pLL/m was formed when increasing the [amine groups in PLys]/[phosphate groups in mRNA] (N/P) ratio above 3. Thus, N/P 4 was used to prepare gluc loading PEG-pLL/m. PEG-pLL (CAA) block copolymer is designed to serve a dual function, electrostatic and covalent interactions for effective loading of mRNA. In acidic pH conditions (pH 3-4), PEG-pLL (CAA) will most likely come in free polymer form with the protonated amines and the closed CAA rings. When this solution was dropped to alkaline mRNA solution (pH 7-8), the residual amine groups in the p(LL-CAA) block can elicit ion complexation with mRNA. Moreover, the left CAA moieties could react with the primary amines on the PLL-CAA block to form pH-sensitive amide bonds with additional adding of polymer. A further reaction between the CAA groups with the unreacted amine groups can cross-link the core of the micelles. To prove CAA's effect, N/P 4 was used in PEG-pLL (CAA)/m. The size of two micelles were examined by dynamic light scattering (DLS), both PEG-pLL and PEG-pLL (CAA) formed micelles of approximately 80 nm in diameter (FIG. 4b). The successful encapsulation of mRNA in the micelles was confirmed by fluorescence correlation spectroscopy (FCS), as the diffusion coefficient of Cy5 labeled mRNA (19.95±1.98 μm2/s) decreased after mixing with the polymers (12.07=0.87 μm²/sec for PEG-pLL/m, 10.94±2.50 μm2/s for PEG-pLL (CAA)/m), which suggested the successful encapsulation of mRNA in micelles. Moreover, by comparing the ratio of counts per molecule between micelle and Cy5-labeled mRNA (Table 1), it was found that two micelles have the analogous number of mRNA per micelle. To further confirm the pH sensitivity of micelles, we evaluated the assemble of micelles by FCS after incubating micelles in different pH buffer (10 mM HEPES buffer with 150 mM NaCl). Here, to better understand the micelle disassemble process, the diffusion coefficient of micelles in different pH buffers was normalized with the initial micelle's diffusion coefficient. In the complexation of PEG-pLL (CAA) and Cy5-mRNA, the normalized diffusion coefficient increased from 1.05 to 1.76 with the decreasing of pH from 6.5 to 4.5, indicating that PEG-pLL (CAA)/m gradually disassociated with Cy5-mRNA as a result of pH decreasing. Also, it was observed that the diffusion coefficient of at PEG-pLL (CAA)/m (19.11±1.33 μm²/sec) was close to that of naked mRNA (19.95±1.87 μm2/sec), which indicated a total release of mRNA at pH 4.5. On the other hand, the normalized diffusion coefficient of PEG-pLL/m remained largely unchanged, suggesting that the PEG-pLL/m kept the association with Cy5-mRNA with the decreasing of pH (FIG. 4c).

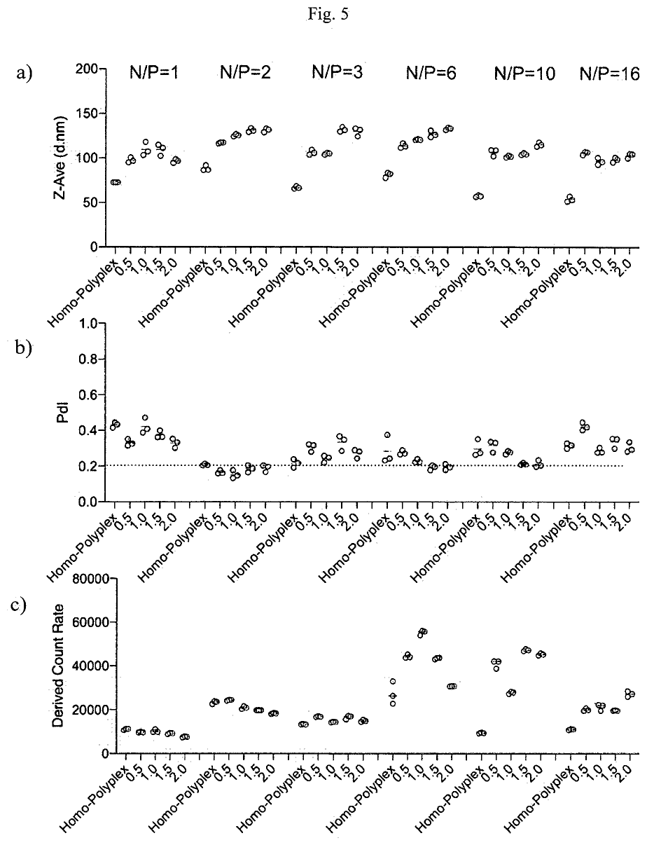

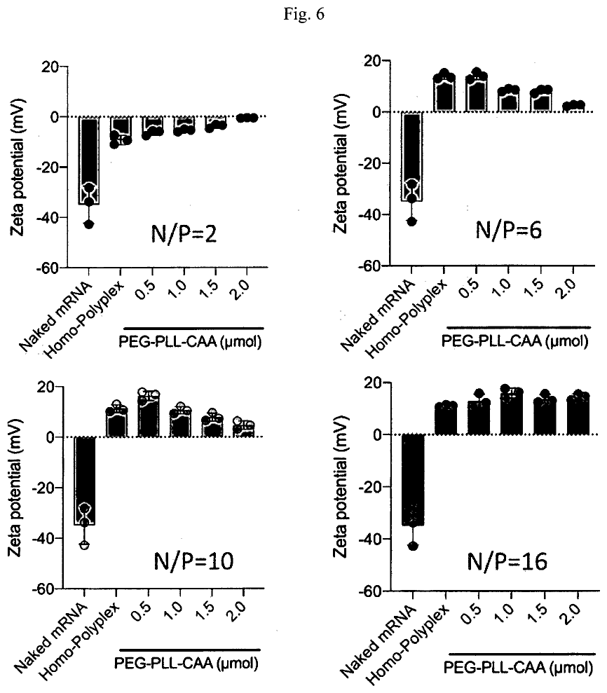

Further, it was found that mRNA polyplex could be also encapsulated by this system. The formation of the micelles was confirmed by DLS (FIG. 5 & FIG. 6).

TABLE 1
Characterization of micelles.

| | | | Diffusion | Count per | Association |
|---|---|---|---|---|---|
| | N/P | Size (nm) | coefficient | molecule | mRNA number |
| Samples | ratio | (mean ± s.d.) ^{a)} | (mean ± s.d.) ^{b)} | (mean ± s.d.) ^{b)} | (mean ± s.d.) ^{b)} |
| Naked | — | — | 19.95 ± 1.98 | 5.82 ± 0.27 | — |
| mRNA | | | | | |
| PEG-pLL/m | 4 | 76 ± 3 | 12.07 ± 0.87 | 7.48 ± 0.79 | 1.36 |
| PEG- | 4 | 83 ± 8 | 10.94 ± 2.50 | 9.85 ± 2.10 | 1.78 |
| pLL(CAA)/m | | | | | |

^{a)} Determined by DLS (n = 3); ^{b)} Determined by FCS (n = 10).

### 3.3 Stability of Micelles

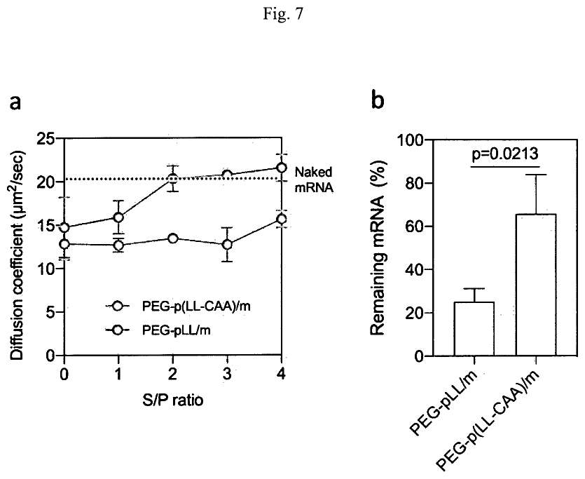

The self-assembly of electrostatic-mediated PICs is known as a reversible process in which nanoparticles coexist with polyions at equilibrium [9,10]. Diluting or adding other charged matter into the system will inherently compromises their integrity, which may cause polyion exchange [11]. Thus, counter polyanion exchange is one of the major concerns for nucleic acid delivery system [12]. Here, we first evaluated the stability of the micelle in the presence of dextran sulfate. Micelles were incubated with dextran sulfate at different S/P([sulfate in dextran sulfate]/[phosphate in mRNA]) ratios for 1 h, the diffusion coefficient of Cy5-mRNA was measured by FCS. In the complexation of PEG-pLL (CAA) and Cy5-mRNA, it was observed that mRNAs were totally released at S/P=2. On the other hand, PEG-pLL (CAA)/m were able to retain mRNA at S/P=4 (FIG. 7a). Besides polyanion exchange, the capability of micelles to protect the loaded mRNA from enzymatic degradation was investigated by qRT-PCR. The results showed that 66% of mRNA were detected in PEG-pLL (CAA)/m group. On the other hand, PEG-pLL/m showed negligible mRNA protection (FIG. 7b). Thus, these results suggest that core cross-link by CAA significantly protected mRNA from polyanion and nuclease attacks, which proves the advantage of this strategy.

### 3.4 In Vitro Activity

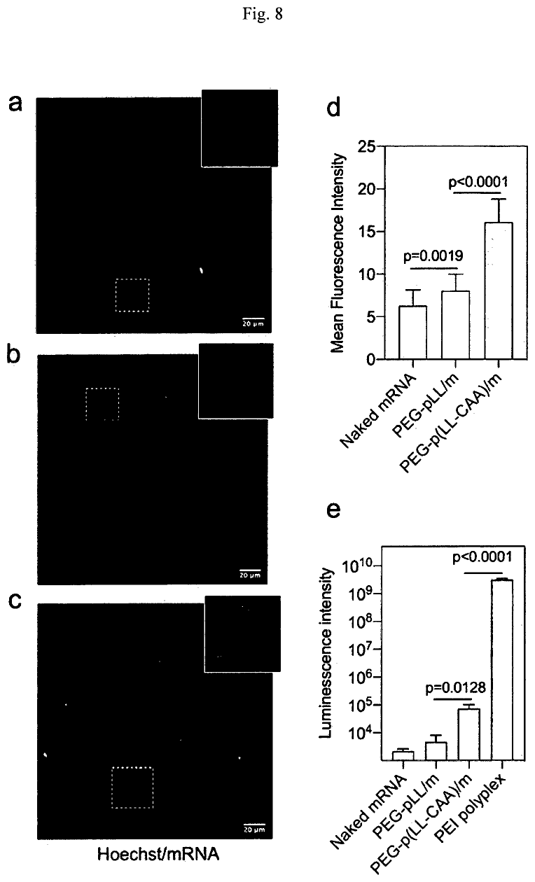

Furtherly, we explored the in vitro performance of mRNA micelles. The cellular uptake was studied in CT 26 cells by using Cy5-labeled mRNA and CLSM. After 6 h incubation, we found that intracellular uptake by can be improved by PIC formation. Moreover, the fluorescence intensity of the Cy5 signal in the cells treated with PEG-pLL (CAA)/m was significantly higher than that of PEG-pLL/m, suggesting improved uptake (FIG. 8a-8d). Next, the protein translation potential was evaluated by the luminescence levels after introduction gluc mRNA to CT 26 cells. As shown in FIG. 8e, PEG-pLL (CAA)/m showed 16-fold increase in gluc expression compared to PEG-pLL/m. It was also noted that the in vitro transfection efficiency of micelles was much lower than that of positive control PEI polyplex.

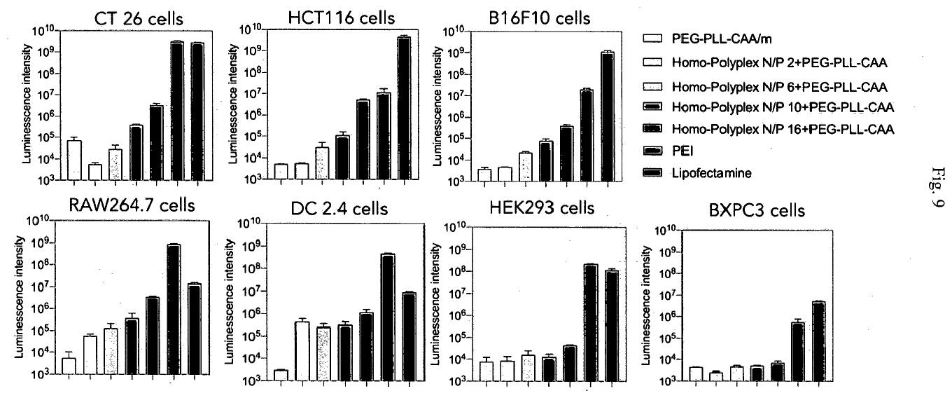

Further, the in vitro transfection efficiency of mRNA polyplex encapsulated PEG-p (LL-CAA) micelle were evaluated in different cell lines. After 24 h incubation, a clear difference in the gluc expression was observed in CT 26 cells, HCT 116 cells, B16F10 cells, RAW246.7 cells, DC2.4 cells (FIG. 9).

### 3.4 In Vivo Activity

Finally, the in vivo mRNA transfection was explored in CT 26 tumor-bearing mice. The mice were treated with a single intratumoral injection of Firefly luciferase (fluc) mRNA, mRNA encapsulated PEG-pLL/m, PEG-pLL (CAA)/m, and also PEI polyplex. The protein expression was traced by measuring the bioluminescence using IVIS at 10 min post-injection of luciferin. Compared with naked mRNA, both PEG-pLL/m and PEG-pLL (CAA)/m exhibited enhanced fluc expression in the injected site at 9 h. Moreover, the luminescence intensity from PEG-pLL (CAA)/m treated group was much higher than that from PEG-pLL/m group (FIG. 10a & FIG. 10c). In contrast, while the control formulation PEI polyplex showed strong gene expression in vitro, it failed to provide a potent gene expression in vivo (FIG. 10).

### Example 2

Pei-Doped mRNA-Loaded Micelles
1. Preparation of PEI-Doped mRNA-Loaded Micelles

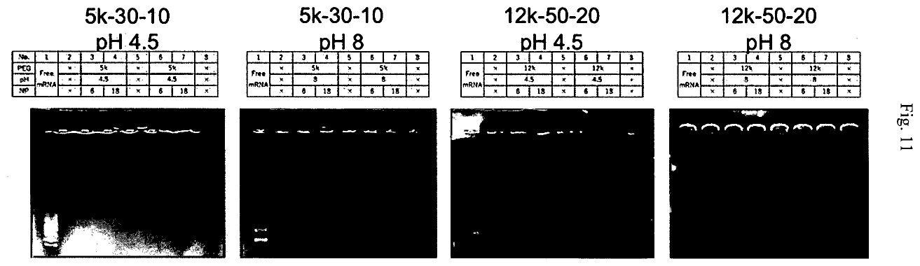

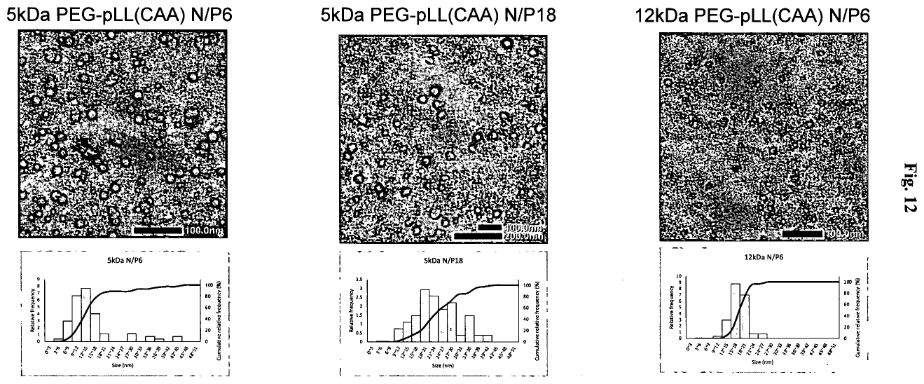

Poly(ethylene glycol)-poly(L-lysine) (PEG-PLL) having a PEG block of 5 kDa or 12 kDa was prepared by ring-opening polymerization of Lysine-NCA. The resulting polymers have a pLL segment of 30 units for the 5 kDa PEG and 50 units for the 12 kDA PEG. The polymers were modified with cis-aconitic anhydride (CAA) to obtain PEG-pLL (CAA). The introduction of CAA in the PLL was 10 units for 5 kDA PEG-PLL and 20 units for 12 kDA PEG-PLL. To prepare the micelles, we first mixed mRNA with branched poly(ethylene imine) (bPEI) at N/P ratio 6 and 18 in 10 mM acetate buffer (pH 4.5). Then, The polyplexes were then mixed with the PEG-PLL (CAA) at 2 mg/ml and the pH was adjusted to pH 8. The size distribution and the zetapotential of the particles was measured by using a Zetasizer NS90 (Table 2). The efficiency of the micelles to load mRNA was confirmed by gel electrophoresis (FIG. 11). The micelles were also observed by transmission electron microscopy (TEM) after staining with uranyl acetate (FIG. 12).

TABLE 2
Average size and zetapotential of
PEI-doped mRNA-loaded micelles

| PEG- | N/P with | | | Polydispersity | Zetapotential |
|---|---|---|---|---|---|
| PLL(CAA) | PEI | pH | Size (nm) | index | (mV) |
| 5k-30-10 | 6 | 4.5 | 73 | 0.319 | — |
| | | 8 | 55 | 0.306 | 0.401 |
| | 18 | 4.5 | 176 | 0.43 | — |
| | | 8 | 63 | 0.38 | 4.86 |
| 12k-50-20 | 6 | 4.5 | 132 | 0.418 | — |
| | | 8 | 80 | 0.368 | −0.227 |
| | 18 | 4.5 | 126 | 0.378 | — |
| | | 8 | 83 | 0.333 | 0.463 |

### 2. In Vitro Activity

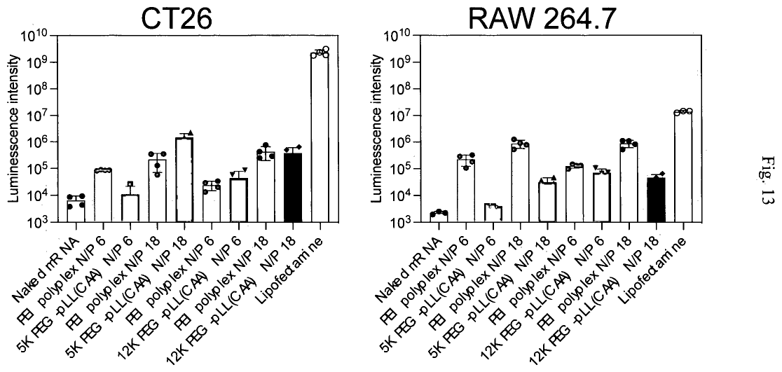

Murine colon adenocarcinoma CT26 cells and RAW 264.7 macrophages were cultured in RPMI containing 10% FBS and 1% penicillin/streptomycin with 5% CO₂ at 37° C. To evaluate Gluc expression efficiency, cells were seeded on a 96-well plate (50,000 cells/well). After 24 h incubation, Gluc mRNA, PEI polyplexes, the PEI-doped mRNA-loaded micelles containing 500 ng of mRNA were applied to the cells. mRNAs were also complexed with lipofectamine and applied to the cells. After another 24 h, a 50 μL culture medium was collected for luciferase assay using a Renilla Luciferase Assay System (Promega, Madison, WI, USA) and a GloMax 96 Microplate Luminometer (Promega, Madison, WI, USA). The results showed differential bioluminescent signals of the PEI-doped mRNA-loaded micelles in the cancer cell and the macrophages (FIG. 13).

1. A pH-responsive carrier for nucleic acid delivery to cells or tissues, comprising a combination of a cationic polymer having a side chain containing a primary amine and a block copolymer represented by the following formula (1): 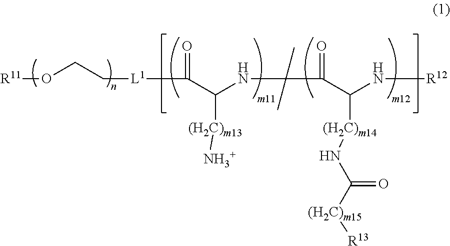
wherein R¹¹ and R¹² each independently represent a hydrogen atom, or an optionally substituted linear or branched alkyl group containing 1 to 12 carbon atoms, or an azide, an amine, maleimide, a ligand or a labeling agent,
R¹³ represents a compound represented by the following formula (I): 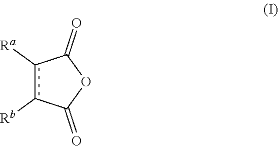
wherein R^{a} and R^{b} each independently represent a hydrogen atom, or an optionally substituted alkyl group, an alkenyl group, a cycloalkyl group, an aryl group, an aralkyl group, an acyl group, a heterocyclic group, a heterocyclic alkyl group, a hydroxy group, an alkoxy group or an aryloxy group, alternatively, R^{a} and R^{b} may be joined with each other to form an aromatic ring or a cycloalkyl ring together with the carbon atoms to which they are attached respectively. The bond between the carbon atoms to which R^{a} and R^{b} are attached respectively may be a single bond or a double bond,
L′ represents NH, CO, or a group represented by the following formula (11): 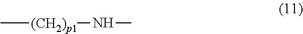
wherein p1 represents an integer of 1 to 6), or
a group represented by the following formula (12): 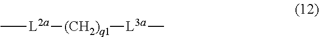
wherein L^{2a} represents OCO, OCONH, NHCO, NHCOO, NHCONH, CONH or COO, L^{3a} represents NH or CO, and q1 represents an integer of 1 to 6,
m11 and m12 each independently represent an integer of 1 to 500 provided that the sum of m11 and m12 represents an integer of 10 to 500), m13, m14 and m15 each independently represent an integer of 1 to 5, and n represents an integer of 1 to 500, and
the symbol “/” means that (m11+m12) units of the respective monomer units shown on the left and right sides of this symbol may be in any sequence.

2. The carrier according to claim 1, wherein the cationic polymer having a side chain containing a primary amine is a polymer represented by the following formula (2), or a branched polyethyleneimine: 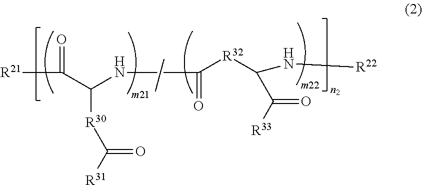
wherein R²¹ and R²² each independently represent a hydrogen atom, or an optionally substituted linear or branched alkyl group containing 1 to 12 carbon atoms, or an azide, an amine, maleimide, a ligand or a labeling agent,
R³⁰ represents (CH₂)ₘ₂₃ (m23 represents an integer of 1 to 5,
R³² represents methylene group or ethylene group,
R³¹ and R³³ each independently represent general formulae (41) or (42) below: 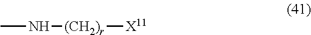
wherein, X¹¹ represents an amine compound residue derived from primary amine compound, and r represents an integer of 0-5, 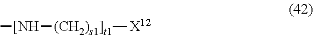
wherein, X¹² is synonymous with X¹¹, and s1 and t1, independently from each other and independently between [NH—(CH₂) s1] units, represent integers of 1-5 and 2-5, respectively,
m21 and m22 each independently represent an integer of 1 to 500 provided that the sum of m21 and m22 represents an integer of 10 to 500,
the symbol “/” means that (m21+m22) units of the respective monomer units shown on the left and right sides of this symbol may be in any Sequence.

3. The carrier according to claim 2, wherein R³¹ and R³³ each independently represent following group:

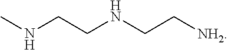

4. The carrier according to claim 1, wherein the compound represented by formula (I) is at least one of compounds represented by the following formulae (Ia) to (Ig):

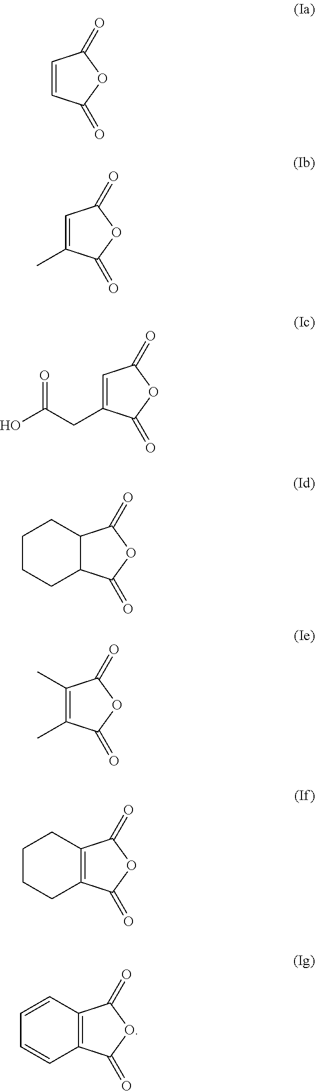

5. The carrier according to claim 4, wherein the compound represented by formula (I) is a compound represented by the following formula (Ia) or (Ib):

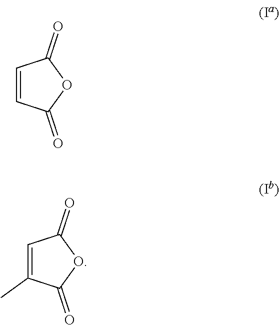

6. The carrier according to claim 1, wherein the block copolymer represented by formula 1 is a block copolymer represented by the following formula (3);

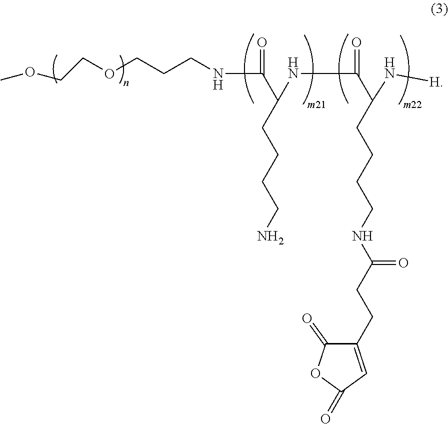

7. A polyion complex comprising the carrier according to claim 1, and nucleic acids.

8. The polyion complex according to claim 7, wherein the cationic polymer having a side chain containing a primary amine is covalently bonded to the block copolymer represented by the formula (1).

9. The polyion complex according to claim 8, wherein the covalent bond is cleaved in a pH-dependent manner.

10. A nucleic acid-delivery kit comprising the polyion complex according to claim 7 for use in nucleic acid delivery to target cells or tissues.

11. A nucleic acid-delivery device comprising the polyion complex according to claim 7 for use in nucleic acids delivery to target cells or tissues.
